(19) United States
(12) Patent Application Publication
(10) Pub. No.: US 2016/0221977 A1
(43) Pub. Date: Aug. 4, 2016

(54) ANTIBACTERIAL BETA-LACTONES, AND METHODS OF IDENTIFICATION, MANUFACTURE AND USE

(71) Applicant: BROWN UNIVERSITY, Providence, RI (US)

(72) Inventors: Jason K. Sello, Providence, RI (US); Corey L. Compton, Providence, RI (US)

(73) Assignee: Brown University, Providence, RI (US)

(21) Appl. No.: 14/914,008

(22) PCT Filed: Aug. 26, 2014
(86) PCT No.: PCT/US2014/052611

§ 371 Date: Feb. 24, 2016

(51) Int. Cl. C07D 305/12

(52) U.S. Cl. CPC C07D 305/12

(57) ABSTRACT

An antibacterial therapeutic

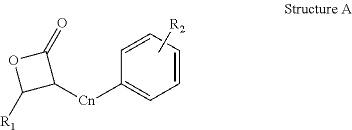

wherein R₁ is selected from the group consisting of alkyl, substituted alkyl, alkenyl, substituted alkenyl, alkynyl, substituted alkynyl, alkoxy, substituted alkoxy, alkoxyalkyl, substituted alkoxyalkyl, NH₂, NHR, NR₂, mono- or polyhydroxy-substituted alkyl, aryl, substituted aryl, heteroaryl, and substituted heteroaryl and the like; and wherein,
R₂ is contemplated as a single substitution of a hydrogen at any position on the benzene ring where the substituted moiety is selected from the group consisting of alkyl, substituted alkyl, alkynyl, substituted alkyl, vinyl, nitro, halo (e.g., includes bromine, chlorine, fluorine and iodine), cyano, aryl, hetero aryl, alkoxy and
Cₙ is carbon and n is a number of from 1 to 5.
The invention further encompasses a method of making Structure A, a pharmaceutical preparation of Structure A and method of therapeutically treating a subject by administering Structure A, with particular reference to treating an infection.

### FIELD OF THE INVENTION

An antibacterial therapeutic

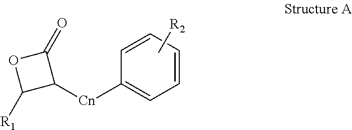

wherein R₁ is selected from the group consisting of alkyl, substituted alkyl, alkenyl, substituted alkenyl, alkynyl, substituted alkynyl, alkoxy, substituted alkoxy, alkoxyalkyl, substituted alkoxyalkyl, NH₂, NHR, NR₂, mono- or polyhydroxy-substituted alkyl, aryl, substituted aryl, heteroaryl, and substituted heteroaryl and the like; and wherein,

R₂ is contemplated as a single substitution of a hydrogen at any position on the benzene ring where the substituted moiety is selected from the group consisting of alkyl, substituted alkyl, alkynyl, substituted alkyl, vinyl, nitro, halo (e.g., includes bromine, chlorine, fluorine and iodine), cyano, aryl, hetero aryl, alkoxy and the like; and

Cₙ is carbon and n is a number of from 1 to 5. The invention further encompasses a method of making Structure A, a pharmaceutical preparation of Structure A and method of therapeutically treating a subject by administering Structure A, with particular reference to treating an infection.

### BACKGROUND OF THE INVENTION

Proteases have been reportedly targeted for the therapeutic treatment of a large family of diseases, including type 2 diabetes, multiple myeloma, HIV, and hepatitis C virus infections. Given the demonstrated physiological significance of this enzyme family in bacterial physiology, proteases have attracted broad interest as potential antibacterial targets. Of special interest are the highly conserved bacterial degradative proteases. Despite the essential physiological roles that these degradative proteases have and their conservation among bacterial species, there are currently no approved antibiotics that target this enzyme class. Disruption of these proteases may diminish cell viability or suppress virulence. Targeting virulence pathways with small molecules could prevent colonization of the host and enable removal of the bacteria by the host immune system. Blocking virulence represents a strategy for the development of novel antibacterial compounds.

The highly conserved, ATP-dependent ClpP (CaseinoLytic Protease) is a specific therapeutic target. ClpP is reported to be essential for virulence in many pathogenic bacteria, including Staphylococcus aureus and Listeria monocytogenes. Thus inhibiting ClpP activity in these organisms could suppress pathogenesis. For example, a clpP null strain of the pathogen Staphylococcus aureus exhibits strongly decreased production of virulence associated hemolysins, lipases, nucleases, and proteases. While ClpP is often required for virulence, it is not essential for viability in most bacteria. However, in the Actinomycetes, a functional ClpP protease is required for viability. This class includes many clinically relevant pathogens such as Mycobacterium tuberculosis, Mycobacterium leprae, Streptomyces, and Corynebacterium. Inhibition of the ClpP protease in these organisms should represent a toxic event, and thus provides an excellent drugable target.

The acyl depsipeptides (ADEPs) are reportedly the only modulators of the essential mycobacterial ClpP peptidase, but M. tuberculosis has efflux pumps that confer resistance.

Note is made of the following publications. The teachings of these publications and all references cited herein are incorporated by reference in their entirety.
Compton, C. L., Schmitz, K. R., Sauer, R. T. Sello, J. K. (2013) “Antibacterial of and Resistance to Small Molecule Inhibitors of the ClpP Peptidase”. ACS Chemical Biology, Epub Ahead of Print. PMID: 24047344
Böttcher T, Sieber S. (2008) β-lactones as specific inhibitors of ClpP attenuate the production of extracellular virulence factors of Staphylococcus aureus. J. Am. Chem. Soc. 130, 14400-14401.
Böttcher T, Sieber S. (2009) β-lactones decrease the intracellular virulence of Listeria monocytogenes in macrophages. ChemMedChem. 4, 1260-1263.
Böttcher T, Sieber S. (2009) Structurally refined β-lactones as potent inhibitors of devastating bacterial virulence factors. ChemBioChem. 10, 663-666.
Danheiser R, Nowick J. (1991) A practical and efficient method for the synthesis of β-lactones. J. Org. Chem. 56, 1176-1185.

### SUMMARY OF THE INVENTION

Disclosed are β-lactones believed to be covalent inhibitors of the ClpP protease. Particularly noted is inhibition of such protease in Escherichia coli, Streptomyces coelicolor, Mycobacterium smegmatis, and Mycobacterium tuberculosis. In addition to the ClpP inhibitors, disclosed herein is a high throughput system to identify new classes of bacterial ClpP activity modulators.

This invention includes a composition of Structure 1

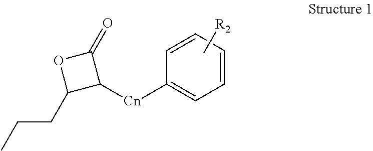

wherein R₂ is a single substitution of a hydrogen at any position on the benzene ring where the substituted moiety is selected from the group consisting of alkyl, substituted alkyl, alkynyl, substituted alkyl, vinyl, nitro, halo (e.g., includes bromine, chlorine, fluorine and iodine), cyano, aryl, hetero aryl, alkoxy; and

Cₙ is carbon and n is a number of from 1 to 5.

In some embodiments, the composition is substantially in cis form or substantially in trans form or a mixture thereof.

Particular embodiments comprise the composition said cis trans mixture wherein the mixture is from about 1:99 cis to trans about 99:1 cis to trans (w/w) with particular reference to about 50:50.

The invention further includes the composition of Structure A

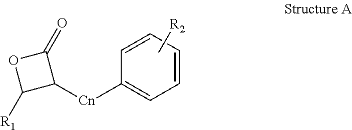

wherein R₁ is selected from the group consisting of alkyl, substituted alkyl, alkenyl, substituted alkenyl, alkynyl, substituted alkynyl, alkoxy, substituted alkoxy, alkoxyalkyl, substituted alkoxyalkyl, NH₂, NHR, NR₂, mono- or polyhydroxy-substituted alkyl, aryl, substituted aryl, heteroaryl, and substituted heteroaryl;

R₂ is a single substitution of a hydrogen at any position on the benzene ring where the substituted moiety is selected from the group consisting of alkyl, substituted alkyl, alkynyl, substituted alkyl, vinyl, nitro, halo, cyano, aryl, hetero aryl, alkoxy; and,

Cₙ is carbon and n is a number of from 1 to 5.

Specific forms of the composition of Structure A noted are

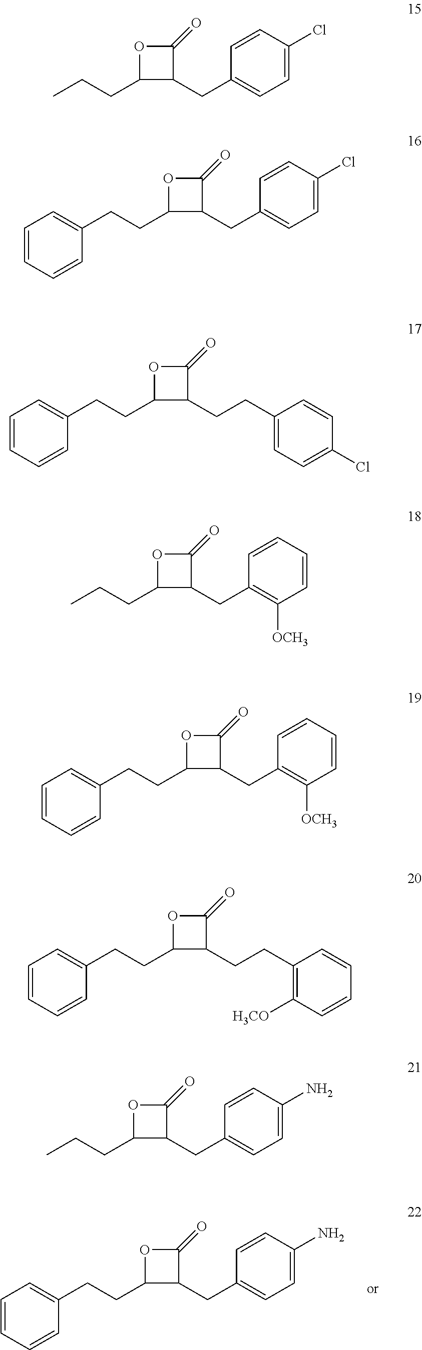

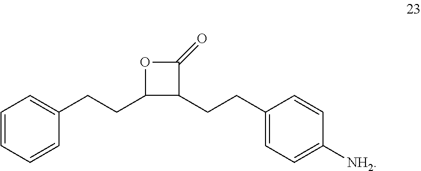 [SMILES: Nc1ccc(CCC2C(=O)OC2CCc2ccccc2)cc1]

In some embodiments, the composition of Structure A is substantially in cis form or substantially in trans form or a mixture thereof.

Particular embodiments comprise the composition said cis trans mixture wherein the mixture is from about 1:99 cis to trans about 99:1 cis to trans (w/w) with particular reference to about 50:50.

Yet further included is a pharmaceutical dosage form comprising a therapeutically effective amount of Structure 1 or Structure A in a pharmaceutically acceptable carrier with particular reference to a topical carrier.

Also included is a method of treating a dermatological infection by the step of topically applying a therapeutically amount of the composition of Structure 1 or Structure A. In particular embodiments of the method the infection treated is selected from the group comprising Staphylococcus aureus, Listeria monocytogenes, Mycobacterium tuberculosis, Mycobacterium leprae, Streptomyces, and Corynebacterium.

The disclosed method of treating a dermatological infection includes the step of topically applying a therapeutically amount of the composition of Structure 1 or Structure A. Particular reference is made to such method in treating infection wherein said infection treated is from the group comprising Staphylococcus aureus, Listeria monocytogenes, Mycobacterium tuberculosis, Mycobacterium leprae, Streptomyces, and Corynebacterium.

### DETAILED DESCRIPTION OF THE INVENTION

Attention is first directed to the disclosed high-throughput cell based assay for discovering modulators of the ClpP protease. The disclosed cell-based assay is useful in the discovery and evaluation of small molecules that modulate ClpP activity.

High-Throughput Cell Based Assay Streptomyces bacteria are reported to have redundant copies of the ClpP protease, encoded by genetic loci clpP1clpP2 and clpP3clpP4. clpPlclpP2 is constitutively transcribed and gives rise to the ClpP1P2 protease. The second bicistronic operon clpP3/4 is not transcribed. Transcription of clpP3clpP4 is reportedly dependent on the transcriptional regulator, PopR. Under normal physiological conditions, ClpP1P2 degrades PopR. If the activity of ClpP1P2 is perturbed, PopR is no longer degraded and accumulates in the cell. Eventually, PopR reaches a concentration in which it binds the PopR dependent promotors and initiates transcription. Small molecules that modulate the activity of ClpP are believed likely to interfere with the ability of ClpP to degrade PopR. Accumulation of PopR in these situations results in concomitant up regulation of transcripts associated with PopR dependent promoters. Indeed, we have found that β-lactones inhibitors of ClpP1P2 induce the transcription of clpP3clpP4 in wild-type S. coelicolor.

A high throughput screen to identify other classes of inhibitors of the ClpP protease is useful. Using a chemical genetics approach, we have constructed S. coelicolor strains that harbor reporter plasmids in which either the neo gene (kanamycin resistance) or the lux gene operon (bioluminescence) are under the control of the PopR-dependent promoter of clpP3clpP4. For the neo reporter (kanamycin resistance), viability of the strain is linked to the transcription of the neo gene, which is under the control of a PopR dependent promotor. Normally ClpP1P2 is degrading PopR, so there is no transcription from the PopR dependent promotors and thus no viability in the presence of Kanamycin. However, when the gene encoding ClpP1P2 have loss of function mutations or the enzyme's activity is inhibited by small molecules, the organism is unable to degrade PopR and is subsequently viable in the presence of kanamycin. Similarly, In the case of the lux reporter (bioluminescence), bioluminescence of the strain is under the same control as the neo reporter. Thus only strains with perturbations in ClpP1P2 activity will show any bioluminescence or resistance to kanamycin.

These reporter strains are useful to identify compounds that interfere with the degradation of PopR by ClpP1P2. Without being bound by any particular theory it is believed that the compounds identified in this assay act by one of three mechanisms: (1) they inhibit the activity of the protease (e.g., β-lactones); (2) they alter the substrate specificity of ClpP (e.g., antibiotic acyldepsipeptides or ADEPs); or (3) they inhibit its activity by binding to its accessory ATPase (e.g., cyclomarin A1).

This reporter strain is believed superior to in vitro reporters in that this assay accounts for permeability, efflux, as well as degradation by native enzymes, such as esterases or proteases.

### EXAMPLE 1

The utility of this reporter strain has been validated with a myriad of small molecule modulators, including 20+β-lactones and 10 ADEPS. Experiments, including positive and negative controls, were performed and the assay correctly identified all activity modulators. Additionally, a concentration dependence of the reporter has been established. As the concentration of β-lactones is increased, so is the density of cell growth with regards to the neo reporter strain. Using this reporter strain we can identify the efficacy of the small molecule for perturbing the ClpP protease activity.

### Procedure for Using Neo Reporter Strain

A micromolar concentration a Structure A or Structure 1 molecule is prepared in DMSO. This DMSO stock is added to molten DNAgar containing 10 μg/mL kanamycin to obtain the desired concentration of small molecule. The agar is plated and allowed to solidify. After solidification, 1×10⁶ spores of wild-type S. coelicolor M145 harboring the neo reporter vector are spread out on top of the media. Optical density is measured at 48 hrs. Known β-lactones inhibitors of ClpP (including Structure) resulted in viability of the strain on the above media conditions. Beta-lactones that do not inhibit ClpP did not enable the reporter strain to grown in media supplemented with kanamycin.

A library of 12 different compounds with a common β-lactone core (compounds 2-13) was synthesized. Two commercially available compounds (β-lactones 1 and 14) were purchased. The bioactivities of all 14 compounds were tested using the S. coelicolor strain with the neo reporter gene. Four compounds (compounds 2, 3, 4, and 7) enabled the reporter strain to grow on media supplemented with kanamycin. These four compounds were presumed to be inhibitors of ClpP1P2.

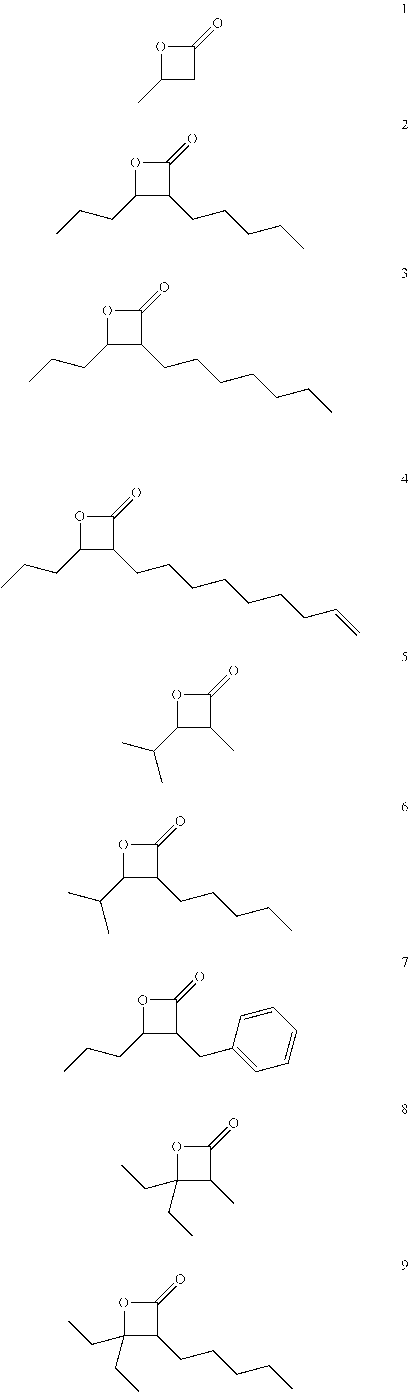

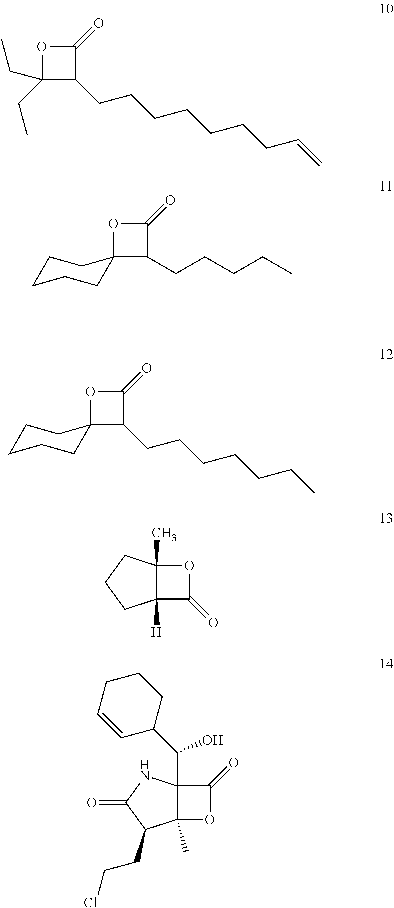

To determine if the results in the S. coelicolor based assay had relevance to Mycobacteria, all fourteen compounds were tested for toxicity to M. smegmatis MC2155, a surrogate of M. tuberculosis. Compounds 2, 3, 4, and 7 were bioactive compounds identified in the S. coelicolor assay and were the compounds that suppressed the growth of M. smegmatis. Three of these inhibitory compounds were trans-disubstituted β-lactones with unbranched alkyl substituents on both the α- and β-carbons (β-lactones 2-4). For these molecules, there was a positive correlation between the length of the alkyl group on the α-carbon and antibacterial activity. However, the more potent compound (β-lactone 7; MIC=10 μg/mL) had an aromatic benzyl substituent on the α-carbon and an unbranched n-propyl group on the β-carbon. The two most active compounds by our test method (4 and 7) from the activity assays with M. smegmatis were subsequently tested for toxicity to the human pathogen, M. tuberculosis H37Rv. Strikingly, both molecules also inhibited growth of M. tuberculosis H37Rv, with β-lactone 7 being the most active against M. smegmatis and M. tuberculosis (MIC=10 and 28 μg/mL, respectively). The compounds were subsequently shown to be inhibitors of ClpP in vitro. In Mycobacteria, ClpP inhibition is associated with killing. Previous reports of ClpP inhibition generally described suppression of virulence in pathogenic bacteria (S. aureus and L. monocytogenes).

Preparation of Antibacterial B-Lactones

Herein we indentify β-lactone covalent inhibitors of the ClpP protease in Escherichia coli, Streptomyces coelicolor, as well as Mycobacterium smegmatis. These compounds are typically toxic to M. smegmatis below 5 μM.

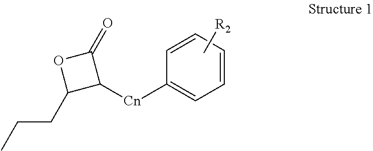

The compound of Structure 1 exists in either cis or trans forms with respect to relative configurations at the alpha and beta carbons of the beta-lactone. With a high performance chromatography instrument, the diastereomers can be separated. Alternatively, a stereospecific synthesis can be used to obtain the pure cis or trans material. The synthetic methods have been reported by Danheiser and co-workers. Danheiser R, Nowick J. (1991) “A practical and efficient method for the synthesis of β-lactones,” The Journal of Organic Chemistry. 56(3):1176-85.

### Preparation of Structure 1

A compound of Structure 1 was prepared by the following means. First, a thiophenol ester was generated from the corresponding acid chloride following the below procedure. After the thiol ester was generated, it was used in a reaction with an appropriate aldehyde to generate Structure 1.

Particular attention is drawn to the following β-lactones as inhibitors of the ClpP peptidase including the peptidase as found in bacteria.

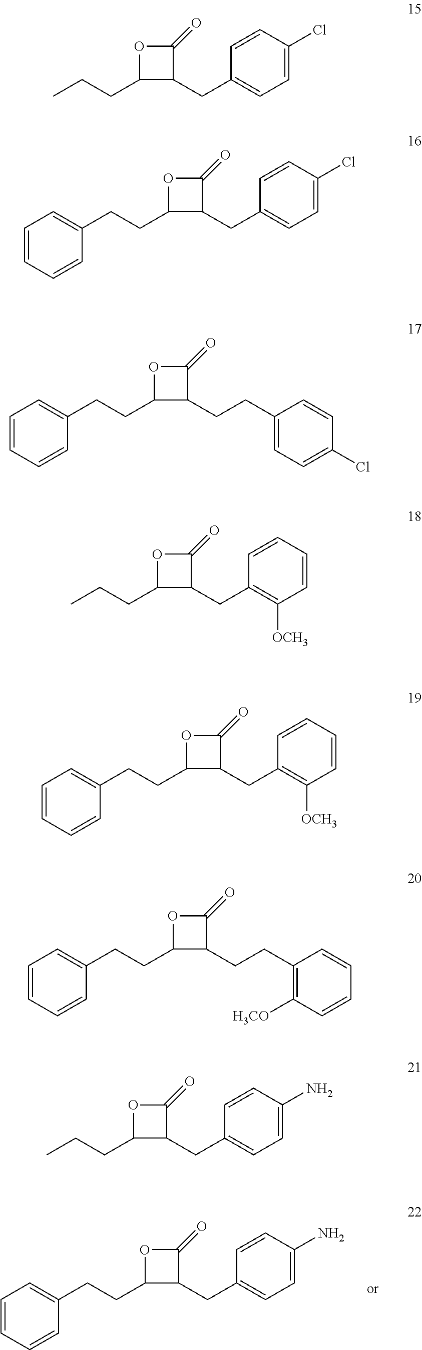

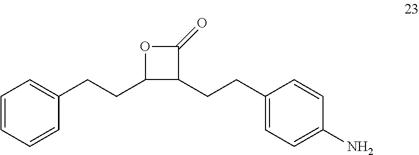 [SMILES: Nc1ccc(CCC2C(=O)OC2CCc2ccccc2)cc1]

These compounds and analogs thereof can be prepared in a straightforward fashion using methodology as disclosed in Danheiser et al. as noted above. In this method, thiol esters are condensed with aldehydes in a base-catalyzed aldol-type reaction. The aldol product of the condensation undergoes a spontaneous cyclization yielding the desired β-lactones. Additional methodology is disclosed in Compton et al. ACS Chemical Biology, as noted above. Analogs of this invention are selected from the exemplified examples or pharmaceutically acceptable salts formed by the reaction of acid and base, such as hydrochloric acid, fumaric acid, maleic acid, succinic acid, acetic acid, citric acid, tartaric acid, carbonic acid, phosphoric acid, oxalic acid, sodium carbonate, sodium hydride, potassium hydroxide, ammonium hydroxide, etc.

### General Procedure for the Preparation of Thiol Esters.

A 100-mL, round-bottomed flask equipped with a rubber septa was evacuated and put under a positive pressure of Nitrogen. Then, charged with 50 mL of methylene chloride, thiophenol (1 eq, 50 mmol), and pyridine (1 eq, 50 mmol) and then cooled in an ice bath while the appropriate acid chloride (1 eq, 50 mmol) was added by syringe over 5 min. The resulting suspension of white solid was stirred for an additional 5 min at 0° C. and at room temperature for 30 min and then poured into 100 mL of dH₂O. The aqueous phase was separated and extracted with 25 mL of methylene chloride, and the combined organic phases were dried over Na₂SO₄, filtered, and concentrated. Flash Chromatography on silica with a mobile phase of 15:1 Hex: EtOAc afforded the desired thiol ester as a colorless oil. Thiol esters were prepared by means of this procedure exhibited an 86-98% yield.

General Procedure for the Preparation of β-Lactones from Thiol Esters and Aldehydes: Preparation of trans 4-butyl 3-pentyloxetan-2-one.

A 100-mL, round-bottomed flask equipped with a rubber septa was evacuated and put under a positive pressure of Nitrogen. The flask was charged with 50 mL of Dry THF and diisopropylamine (1.1 eq, 11 mmol), and then cooled in an dry ice and acetone bath (−78° C.) while n-butyllithium solution (2.25 M in hexanes, 1.035 eq, 10.35 mmol) was added via syringe over 2 min. After 30 min, S-phenyl 3-phenylpropanethioate (1 eq, 10.0 mmol) was added drop wise via syringe over 5 min. After 30 min, a solution of butyryl aldehyde (1 eq, 10.0 mmol) in 12.5 mL of THF was added dropwise over 20 min via a syringe cooled externally with dry ice. The reaction mixture was stirred at −78 ° C. for 60 min and then allowed to warm to 0° C. over 120 min. Saturated NH₄Cl solution (50 mL) was then added, and the resulting mixture was partitioned between 50 mL of water and 50 mL of diethyl ether. The organic phase was extracted with three 50-mL portions of 1N HCL, three 50-mL portions of sat. Na₂CO₃ solution and two 50-mL portions of saturated NaCl solution, dried over NaSO₄, filtered, and concentrated to afford a pale yellow oil. Flash column chromatography on silica gel (gradient elution with ethyl acetate: hexanes) gave single diastereomeric β-Lactones.

### EXAMPLE 3

### Therapeutic Use of Structure 1

The above compounds or derivatives are used to treat bacterial infections caused by number of pathogenic bacteria, including but not limited to Mycobacterium, Streptomyces, Listeria, Corynebacterium, Staphylococcus, and Streptococcus. The compounds could be applied as a topical cream, capsule or tablet, or as an injection. Treatment with the compounds can directly cause selective toxicity or avirulence in these pathogenic strains.

A 49 year-old overweight male presents a wound on the skin that is infected with Mycobacterium leprae, Streptococcus aureus, etc. The subject is treated with a topical dosage of cis Structure 1 at 3mg/kg of ointment (White Petrolatum and Mineral Oil). The infection is resolved within 12 hours.

In another embodiment the ointment of structure 1 contains 0.35% Structure 1 by weight.
Therapeutically effect ranges of Structure 1 are (i) for cis, (ii) for trans and (iii) for a mixture—from about 0.001 mg/kg to about 100 mg/kg. The compounds are administered as an antibacterial ointment. Further formulations are known in the art with particular reference to Marriott, et al., Pharmaceutical Compounding and Dispensing, (Pharmaceutical Pr; 2 edition (2010)), ISBN-10: 0853699127.

The pharmacologically active compositions of this invention can be processed in accordance with conventional methods of Galenic pharmacy to produce medicinal agents for administration to subjects, e.g., mammals including humans.

The compositions of this invention individually or in combination are employed in admixture with conventional excipients, i.e., pharmaceutically acceptable organic or inorganic carrier substances suitable for parenteral, enteral (e.g., oral or inhalation) or topical application which do not deleteriously react with the active compositions. Note is made of ointment bases including
Hydrocarbon bases, e.g. hard paraffin, soft paraffin, microcrystalline wax and ceresine;
Absorption bases, e.g. wool fat, beeswax;
Water soluble bases, e.g. macrogols 200, 300, 400;
Emulsifying bases, e.g. emulsifying wax, cetrimide; and,
Vegetable oils, e.g. olive oil, coconut oil, sesame oil, almond oil and peanut oil.

Suitable pharmaceutically acceptable carriers include but are not limited to water, salt solutions, alcohols, gum arabic, vegetable oils, benzyl alcohols, polyethylene glycols, gelatin, carbohydrates such as lactose, amylose or starch, magnesium stearate, talc, titanium dioxide, silicic acid, viscous paraffin, perfume oil, fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compositions. They can also be combined where desired with other active agents, e.g., vitamins.

In some embodiments of the present invention, dosage forms include instructions for the use of such compositions.

For parenteral application, particularly suitable are injectable, sterile solutions, preferably oily or aqueous solutions, as well as suspensions, emulsions, or implants, including suppositories. Ampules, vials, and injector cartridges are convenient unit dosages.

Also for parenteral application, particularly suitable are tablets, dragees, liquids, drops, suppositories, or capsules. A syrup, elixir, or the like can be used wherein a sweetened vehicle is employed. Sublingual and buccal forms are also noted.

Sustained or directed release compositions can be formulated, e.g., liposomes or those wherein the active component is protected with differentially degradable coatings, e.g., by microencapsulation, multiple coatings, etc. It is also possible to freeze-dry the new compositions and use the lyophilizates obtained, for example, for the preparation of products for injection.

1. The composition 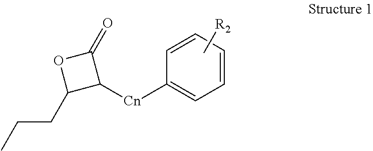
R₂ is a single substitution of a hydrogen at any position on the benzene ring where the substituted moiety is selected from the group consisting of alkyl, substituted alkyl, alkynyl, substituted alkyl, vinyl, nitro, halo (e.g., includes bromine, chlorine, fluorine and iodine), cyano, aryl, hetero aryl, alkoxy;
Cₙ is carbon and n is a number of from 1 to 5, and analogs thereof.

2. The composition of claim 1 substantially in cis form.

3. The Composition of claim 1 substantially in trans form.

4. The composition of claim 1 in a mixture of cis and trans form.

5. The composition of claim 4 wherein said mixture is from about 1:99 cis to trans about 99:1 cis to trans (w/w).

6. The composition of claim 5 wherein said cis to trans is about 50:50.

7. The composition 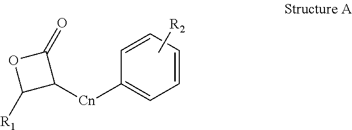
wherein R₁ is selected from the group consisting of alkyl, substituted alkyl, alkenyl, substituted alkenyl, alkynyl, substituted alkynyl, alkoxy, substituted alkoxy, alkoxyalkyl, substituted alkoxyalkyl, NH₂, NHR, NR₂, mono- or polyhydroxy-substituted alkyl, aryl, substituted aryl, heteroaryl, and substituted heteroaryl;
R₂ is c a single substitution of a hydrogen at any position on the benzene ring where the substituted moiety is selected from the group consisting of alkyl, substituted alkyl, alkynyl, substituted alkyl, vinyl, nitro, halo, cyano, aryl, hetero aryl, alkoxy; and,
Cₙ is carbon and n is a number of from 1 to 5.

8. The composition of claim 7 wherein said composition is selected from the from the group comprising 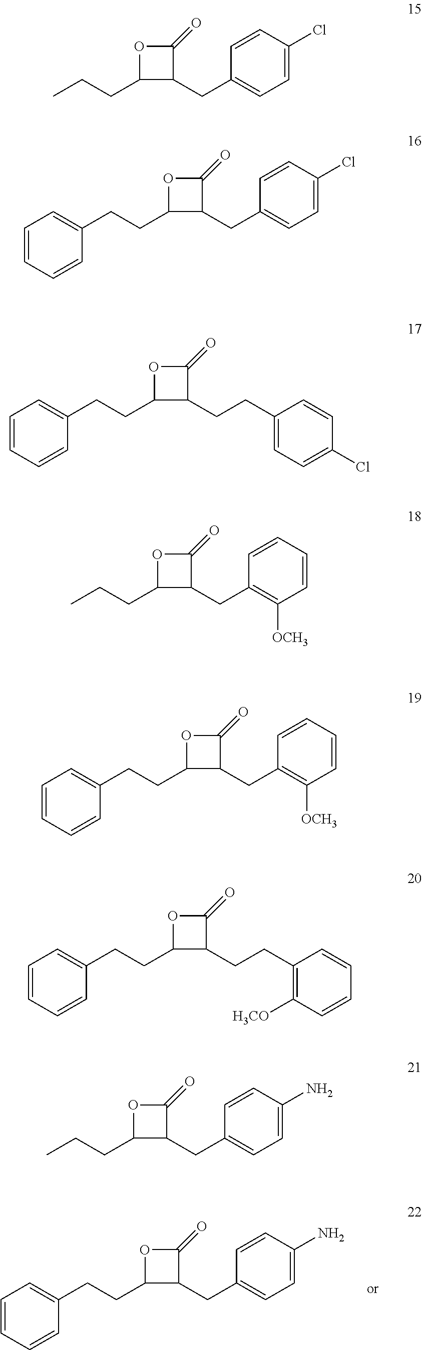 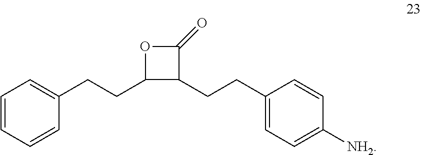 [SMILES: Nc1ccc(CCC2C(=O)OC2CCc2ccccc2)cc1]
and analogues thereof.

9. The composition of claim 7 substantially in cis form.

10. The Composition of claim 7 substantially in trans form.

11. The composition of claim 7 in a mixture of cis and trans form.

12. The composition of claim 11 wherein said mixture is from about 1:99 cis to trans about 99:1 cis to trans (w/w).

13. The composition of claim 12 wherein said cis to trans is about 50:50.

14. A pharmaceutical dosage form comprising a therapeutically effective amount of Structure 1 in a pharmaceutically acceptable topical carrier.

15. A pharmaceutical dosage form comprising a therapeutically effective amount of Structure A in a pharmaceutically acceptable topical carrier.

16. A method of treating a dermatological infection by the step of topically applying a therapeutically amount of the composition of claim 1.

17. The method of claim 16 wherein said infection is selected from the group comprising Staphylococcus aureus, Listeria monocytogenes, Mycobacterium tuberculosis, Mycobacterium leprae, Streptomyces, and Corynebacterium.

18. A method of treating a dermatological infection by the step of topically applying a therapeutically amount of the composition of claim 7.

19. The method of claim 18 wherein said infection is selected from the group comprising Staphylococcus aureus, Listeria monocytogenes, Mycobacterium tuberculosis, Mycobacterium leprae, Streptomyces, and Corynebacterium.
